(19) United States
(12) Patent Application Publication
(10) Pub. No.: US 2025/0125012 A1
(43) Pub. Date: Apr. 17, 2025

(54) TUMOR NEOANTIGEN PREDICTION METHOD AND TUMOR NEOANTIGEN PREDICTION SYSTEM

(71) Applicant: Acer Incorporated, New Taipei City (TW)

(72) Inventors: Chi-Wei Lu, New Taipei City (TW); Ying-Ja Chen, Taipei City (TW); Li-Tzu Yeh, New Taipei City (TW); Tao-Chuan Shih, Taipei City (TW); Cing-Han Yang, Keelung City (TW); Tun-Wen Pai, Taipei City (TW)

(73) Assignee: Acer Incorporated, New Taipei City (TW)

(21) Appl. No.: 18/504,127

(22) Filed: Nov. 7, 2023

(30) Foreign Application Priority Data
Oct. 16, 2023 (TW) 112139360

(51) Int. Cl. G16B 40/00; G16B 20/20; G16B 30/10

(52) U.S. Cl. CPC G16B 40/00; G16B 20/20; G16B 30/10

(57) ABSTRACT

A tumor neoantigen prediction method and a tumor neoantigen prediction system are provided. In the method, multiple amino acid sequences in genes of a person to be tested are extracted as multiple test peptides to be compared with multiple human protein sequences in a protein sequence database to find multiple similar peptides that match the human protein sequences. The similar peptides are filtered out from the test peptides and the filtered test peptides are input to multiple trained human leukocyte antigen (HLA) models to obtain multiple ranking results of the test peptides. A weighted sum of rankings of each test peptide in the ranking results is calculated as a score of the test peptide. At least one of the test peptides is selected as a neoantigen adapted for the person to be tested according to the score.

### CROSS-REFERENCE TO RELATED APPLICATION

This application claims the priority benefit of Taiwan application serial no. 112139360, filed on Oct. 16, 2023. The entirety of the above-mentioned patent application is hereby incorporated by reference herein and made a part of this specification.

### BACKGROUND

### Technical Field

The disclosure relates to a prediction method and system, and in particular to a tumor neoantigen prediction method and a tumor neoantigen prediction system.

### Description of Related Art

Neoepitopes and pathogen peptides are fundamental players in the complex interaction between the immune system and diseases. Neoepitopes resulting from amino acid alterations have the ability to be recognized by the immune system as abnormal, thereby triggering a targeted immune response against cancers. The neoepitopes are derived from tumor-specific antigens

(TSAs), which are different from tumor-associated antigens (TAAs) produced by abnormal expression within cancerous cells. In contrast, pathogen peptides play a pivotal role in immune responses against infectious agents. As invading pathogens replicate within the host, various proteins are produced. Some of the proteins are processed into peptides and presented by major histocompatibility complex (MHC) molecules to T cells. T cells are essential effectors of the adaptive immune system.

When a T-cell receptor (TCR) binds to an antigen-MHC complex, the T-cell receptor activates the T cell and triggers proliferation and differentiation thereof, which leads to the formation of effector T cells that can directly eliminate cells presenting the antigen and memory T cells that remember antigens for faster and stronger responses upon re-exposure. The diversity and specificity of TCRs are due to the unique combination of V, D, and J gene segments in the TCR genes. The V, D, and J recombination during T-cell development results in various TCR a and β chains, allowing the immune system to effectively recognize and respond to various pathogens and abnormal cells.

Accordingly, how to find neoantigens adapted to combat gene mutations of the cancerous cell from amino acid sequences in human genes is a major issue for persons skilled in the art.

### SUMMARY

The disclosure provides a tumor neoantigen prediction method and a tumor neoantigen prediction system, which can find neoantigens adapted to combat gene mutations of cancerous cells.

An embodiment of the disclosure provides a tumor neoantigen prediction method, which includes the following steps. Multiple amino acid sequences in genes of a person to be tested are extracted as multiple test peptides to be compared with multiple human protein sequences in a protein sequence database to find multiple similar peptides that match the human protein sequences. The similar peptides are filtered out from the test peptides and the filtered test peptides are respectively input to multiple trained human leukocyte antigen (HLA) models to obtain multiple ranking results of the test peptides. A weighted sum of rankings of each test peptide in the ranking results is calculated as a score of the test peptide. At least one of the test peptides is selected as a neoantigen adapted for the person to be tested according to the score.

An embodiment of the disclosure provides a tumor neoantigen prediction system, which includes a data extraction device, a storage device, and a processing device. The data extraction device is used to extract multiple amino acid sequences in genes of a person to be tested. The storage device is used to store a protein sequence database recorded with multiple human protein sequences. The processing device is coupled to the data extraction device and the storage device, and is configured to compare the amino acid sequences extracted by the data extraction device as multiple test peptides with the human protein sequences in the protein sequence database to find multiple similar peptides that match the human protein sequences, filter out the similar peptides from the test peptides and respectively input the filtered test peptides to multiple trained human leukocyte antigen models to obtain multiple ranking results of the test peptides, calculate a weighted sum of rankings of each test peptide in the ranking results as a score of the test peptide, and select at least one of the test peptides as a neoantigen adapted for the person to be tested according to the score.

Based on the above, in the tumor neoantigen prediction method and the tumor neoantigen prediction system of the embodiments of the disclosure, self-similarity and pathogen-similarity analyses are performed on the amino acid sequences in the genes of the person to be tested, and in combination with the HLA models and a driver mutation analysis, the neoantigens adapted to combat the gene mutations of the cancerous cells of the person to be tested are found, which can facilitate advances in T-cell immunotherapy and personalized cancer treatment.

In order for the features and advantages of the disclosure to be more comprehensible, the following specific embodiments are described in detail in conjunction with the drawings.

### BRIEF DESCRIPTION OF THE DRAWINGS

FIG. 1 is a schematic diagram of constructing a human biological database according to an embodiment of the disclosure.

FIG. 2 is a schematic diagram of tumor neoantigen prediction according to an embodiment of the disclosure.

FIG. 3 is a block diagram of a tumor neoantigen prediction system according to an embodiment of the disclosure.

FIG. 4 is a schematic diagram of a tumor neoantigen prediction method according to an embodiment of the disclosure.

FIG. 5 is a schematic diagram of an amino acid sequence comparison/alignment method according to an embodiment of the disclosure.

### DESCRIPTION OF THE EMBODIMENTS

In order to promote researches on T-cell immunotherapy and enhance the understanding of antigen-pathological relationships, embodiments of the disclosure consolidate pathogen peptide and neoepitope data with T-cell assay information into a centralized human biological database to provide comprehensive neoepitope data and corresponding T-cell assay information, which facilitate the researches on T-cell immunotherapy and the understanding of antigen-pathological relationships, and provide various functions such as tumor neoantigen prediction and visualization to support pathogen studies. Users can search for sequences targeting human amino acids (self-similarity) or known pathogen protein sequences (pathogen-similarity) with T-cell assay-verified information through sequence alignment.

By curating data from published literatures and existing databases, the embodiments of the disclosure consolidate information such as antigens, major histocompatibility complex (MHC) types, T-cell assays, T-cell receptor (TCR)-peptide-MHC (pMHC) structural data, and mutation types for cancer-related entries in the database to provide a unified and user-friendly platform for researchers in the field of T-cell immunotherapy.

In detail, the database employs, for example, a semi-automatic approach to ensure data reliability and transforms outdated MHC naming conventions to modern equivalent naming conventions, thereby enhancing data accuracy and consistency. The database provides powerful search and visualization functions, enabling researchers to explore and analyze human TCR beta chain sequences associated with specific tissues, clinical conditions, or cell types.

In addition to the basic data integration and visualization functions, the database also provides various analytical tools, which include a string search method (pathogen-similarity and self-similarity analyses), simplifying analysis and classification for species categories by offering high-level/fuzzy grouping, and assigning scores to each data entry based on the completeness of the fields.

Moreover, the database offers motif/peptide analysis and integrates predictive tools for assessing peptide-MHC (pMHC) binding affinity. The database aims to contribute to pathogen-peptide and neoepitope researches in all aspects by providing a reliable and carefully curated resource that can support the neoepitope research community and facilitate advances in T-cell immunotherapy and personalized cancer treatment.

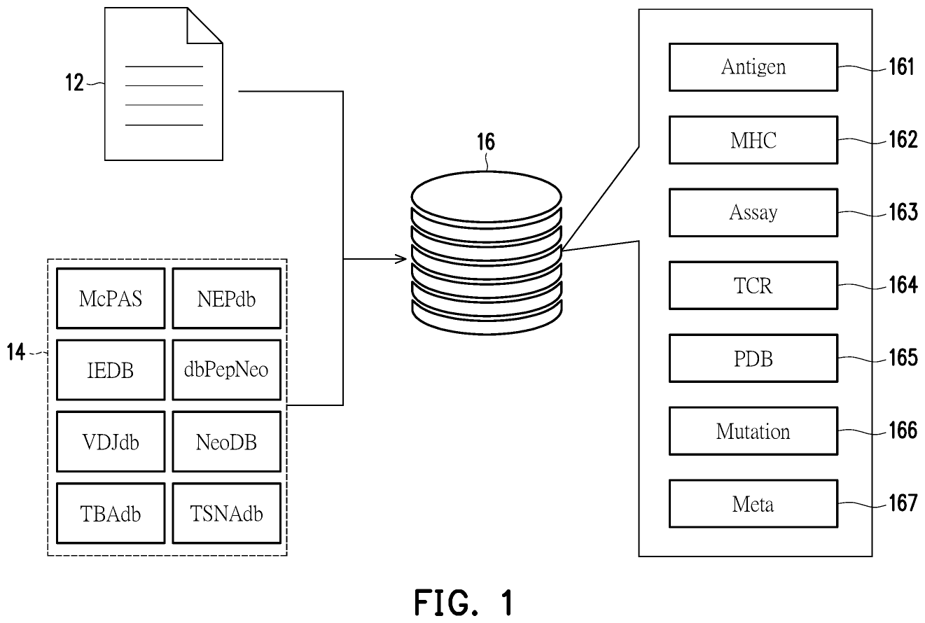

FIG. 1 is a schematic diagram of constructing a human biological database according to an embodiment of the disclosure. Please refer to FIG. 1. The embodiment of the disclosure collects entries from a published citation 12 and an existing pathogen or neoepitope database 14 using a semi-automatic approach to construct a human biological database 16 and ensure that the human biological database 16 includes only data from published sources.

In detail, the embodiment performs PubMed queries using various keywords, including complementarity-determining region (CDR), MHC, neoepitope, and pathogen or cancer type, to search for the citation 12, and filters the obtained literature 12 to retain information that contains pathogen- or neoantigen-related data with experimental validation. The initial filtering criteria involves the presence of peptides, MHC types, disease types, and assay information. Other criteria consider the availability of CDR3 sequences, CDR types, protein data bank (PDB) information, mutation types, wild-type/mutated sequences, and mutated amino acids. Other information (such as classification) is populated using semi-automatic scripts.

On the other hand, the embodiment obtains entries related to human species, infections, cancers, HLA, T-cell assays, neoepitopes, and structural information by querying the pathogen or neoepitope database 14. The pathogen or neoepitope database 14 includes McPAS, IEDB, VDJdb, TBAdb, NEPdb, dbpepNeo, NeoDB, TSNAdb, etc., which are described below.

### [Pathogen Database]

(1) IEDB: The IEDB is a comprehensive database that focuses on immune epitopes and associated assays thereof (such as T-cell assays). In particular, a query is conducted on the IEDB homepage to retrieve linear peptide epitopes that have been tested using T-cell assays and demonstrated positive results. The query excludes B-cell and does not apply any MHC class restriction. The search focuses on human host organisms for infectious diseases.

All peptides collected through the query have T-cell assay information and demonstrate positive T-cell responses. The peptides have undergone experimental evaluation and shown the ability to elicit a T-cell response in the context of infectious diseases. The T-cell assays in the database can be categorized into three main groups: 3D assays, biological activity assays, and binding assays.

In the binding category, for some entries, numerical values are provided to indicate the recognition and responsiveness of T cells to neoepitopes. However, data mis-recordings may occur and lead to inaccurate values.

To address the issue, the embodiment implements a filtering process based on KD values, on-rate, and off-rate distributions, wherein specific thresholds are set to filter out anomalous entries, such as large KD values. After multiple rounds of filtering, entries that meet the criteria are selected to construct a database, ensuring the inclusion of high-quality and reliable data.
(2) VDJdb: VDJdb is a carefully curated database that contains T-cell receptor (TCR) sequences and corresponding antigen specificities thereof. The target of the embodiment is to focus on the peptide-MHC (pMHC) information that accompanies the TCR data. During the selection process, the embodiment filters the database mainly based on the “Species” field and chooses entries related to human species, excluding entries for monkey and mouse. However, it is worth noting that VDJdb also includes entries related to cancers, which are labeled with “Parent Species”, that is, Homo sapiens. The embodiment retains the entries but marks the entries accordingly, providing the corresponding PMID for users to make own judgments. The entries are considered more closely related to neoepitopes rather than pathogens.
(3) McPAS: McPAS-TCR is a manually curated database that focuses on TCR sequence, pathologies, and antigen information. Similar to the approach with VDJdb, the embodiment filters the data in McPAS-TCR to include only entries related to human infections and exclude entries for other species, based on the parental species field.
(4) TBAdb: TCRs and BCRs targeting known antigens (TBAdb) is a manually curated database in Pan Immune Repertoire Database (PIRD). The embodiment obtains the database from the repository of R-language tool immunearch. Compared with VDJdb and McPAS-TCR, the database has more detailed information regarding T-cell assay (sequencing methods and number of experiments, etc.) and a scoring method to determine the reliability of the data. The embodiment also obtains entries only if the host is human and the source of the disease is an infection.

### [Neoepitope Database (NEPdb)]

NEPdb is a comprehensive database that contains more than 17,000 validated entries of human immunogenic and non-immunogenic neoepitopes. The entries provide detailed information on HLAs and corresponding T-cell responses thereof. Only positive results are included in the database, ensuring that the database focuses on only relevant and impactful data. Regarding dbPepNeo, NeoDB, and TSNAdb, no additional filters are applied, and the entries from the databases are trivially adjusted to ensure compatibility and consistency. The TSNAdb is utilized to identify and assess any duplicate occurrences of different data across the aforementioned databases and may be used as a reference to detect instances of data duplication with different databases.

### [Structural Database]

To complement the pathogen and neoepitope databases mentioned earlier, the embodiment also includes collecting structural information from the TCR3d and SterDab databases, and comparing the peptide sequences with pathogen sequences obtained from the structural databases. If the sequences possess 100% sequence identities, duplicate PubMed identifiers (PubMed IDs), and trigger the same MHC, the embodiment marks the entries as “Matched” notation, and assigns corresponding structural data (such as PDB IDs, chains, and TCR information) from the structural database to the entries.

However, if entries happen to have duplicated sequences and MHC information but different PubMed IDs, the embodiment still assigns the structural information but marks the entries as “Ambiguous” for the judgment by the user. For the “Ambiguous” entries, if the pathogen database and the structural database are different, the embodiment provides the results of sequence alignment between the same. For entries without structural information, the embodiment still represents the pMHC and TCR status with a simple graphical representation, which ensures that even without structural data, the relevant pMHC and TCR information may be conveyed to the users.

Each entry in the human biological database 16 is classified into a specific group according to fields such as antigen 161, MHC 162, assay 163, TCR 164, PDB 165, mutation 166, and meta 167, which are described below.

### [Antigen]

The field provides information about the pathological conditions associated with the antigens and the corresponding diseases, including detailed information such as the antigen sequence, gene, and sequence length, and also including the parental species, source molecule, and source organism of the corresponding entries. To simplify the representation of various pathological sources, the embodiment classifies the parental species into the following four main groups:
(1) Virus: further classified into DNA viruses and RNA viruses based on amino acid composition.
(2) Bacteria: includes pathological bacteria, environmental bacteria, and probiotics.
(3) Cancer: entries related to cancer are collected from databases such as dbPepNeo, Neodb, NEPdb, and TSNAdb. It is worth noting that VDJdb also includes entries related to cancers and the entries are labeled as “Homo sapiens”. Although specific cancer types may not be indicated, the embodiment retains the entries and classifies the entries under “Homo sapiens” in the cancer category.
(4) Other: the category primarily describes synthetic or botanical sequences, as well as specific therapeutics for different disease conditions.

The parental species mentioned in the above entries may be further classified into subcategories, and the subcategories are grouped into the four main categories mentioned above. For example, under the Virus category, there may be subcategories such as Hepacivirus, including Hepatovirus A, Hepatitis B, and Heron hepatitis B virus.

### [Major Histocompatibility Complex (MHC)]

The field displays the types of MHC associated with the disease sequences. However, it is worth noting that certain sources may include outdated naming schemes for MHC types. To address the issue, the embodiment creates a conversion table to transform the old naming schemes into more up-to-date naming schemes. Within the field, the embodiment provides four key pieces of information:
(1) The naming as provided by the source databases or literature;
(2) the conversion status: indicates whether a conversion is performed for the given MHC type;
(3) the converted naming: represents the MHC type after undergoing the conversion process;
(4) the converted naming in higher resolution: provides all possible MHC types in a more detailed format after conversion.

### [Assay]

The embodiment categorizes the types of T-cell assays into four broad categories based on reference materials from IEDB:
(1) Biological activity (BA): the category contains various T-cell assays that are primarily used to assess the biologically activity of T cells. The category covers various sorting methods for culturing T cells, such as sorting and culture, and also includes some specific assays such as 51 chromium, 3H-thymidine, etc.;
(2) binding assays: the category focuses on the ability of T cells to bind antigens, including various assays, such as surface plasmon resonance (SPR), limiting-dilution-cloning, tetramer-sort, pentamer-sort, multi-element assay, etc.;
(3) cytokine: the category focuses on cytokines released by T cells and is used to assess the immune response of T cells, including various assays such as cytometric bead array, radio immuno assay (RIA), IFNg capture assay, bioassay, enzyme-linked immunosorbent assay (ELISA), enzyme-linked immunospot (ELISPOT) etc.;
(4) three-dimensional (3D) structure: the category is mainly concerned with the analysis of the three-dimensional structure of T-cell receptors and antigen complexes, mainly using methods such as x-ray crystallography.

Each data entry is categorized into the four major categories mentioned above. The original methods and responses are represented by the “Method” and “Response” fields. For the binding and 3D categories, the data may include qualitative and quantitative measurements to indicate the degree of binding.

### [Protein Database (PDB) and T-Cell Receptor (TCR) Information]

The embodiment establishes a process to compare sequences in the antigen database with sequences in the structural database. If a sequence is found to have a matching sequence, MHC type or PubMed identifier in the structural database, the embodiment fills in the corresponding information from the structural database.

In the field, the embodiment provides the following details from the structural database: PDB identifier (whether a complex structure or a single-chain structure), PDB organism, the PDB PubMed identifier and the publication date, and the antigen, TCR, and MHC chains present in the PDB, including additional information such as CDR3 and VDJ amino acid compositions.

### [Mutation]

For cancer entries, the embodiment provides information on the mutation types, which are categorized as follows: single nucleotide variants (SNV), small insertion of deletions (InDel), copy number variants (CNV), and gene fusion.

If the information is available in the source database, the embodiment may also include detailed information such as the wild type peptide, mutant type peptide, and sequence alignment for the mutated amino acids. The additional fields help to describe the specific mutations and the impact on the peptide sequences in the context of cancer-related entries.

### [Meta]

Since all data is derived from published literature, the field mainly provides the PubMed identifier, title, author, journal information, and the date of publication. Furthermore, the embodiment also includes information about the sequencing methods and the tissues for antigen sequencing from the literature or a specific database. The “Source” field also indicates the database where the sequence is obtained from or if the sequence is collected through own data collection efforts. If a sequence appears in multiple databases, the embodiment adds “Common_source” field to record the databases in which the sequence may be found and adds “Completeness score” based on the level of available information.

Users can search the human biological database to identify and search TCR sequences similar to the target sequences, and filter the search results by selecting various options (such as MHC-type and specific genes), thereby obtaining TCR sequences that meet specific criteria.

For example, for self-similarity, the queried antigen sequence is compared with a protein sequence database such as Uniprot to identify any similarities or matches with the wild-type sequence. The comparison helps to determine if the queried antigen sequence possess any known counterparts or related sequences.

In detail, given the diversity of TCR sequences and the tendency of similar sequences to exhibit similar properties, the embodiment of the disclosure utilizes a similarity measurement approach akin to Levenshtein distance. The method quantifies the minimum number of insertions, deletions, and substitutions required to transform one sequence into another. Users can specify the maximum number of allowance gaps and character replacements, enabling the identification of sequences that are identical or similar to a given test sequence. In some embodiments, the BLAST algorithm is used to perform string matching on human protein sequences in a protein sequence database, and the strings are filtered based on specific criteria. The criteria include sequence search, sequence similarity, sequence length, gap, and character substitutions. For example, users can specify the permissible number of gaps and character replacements.

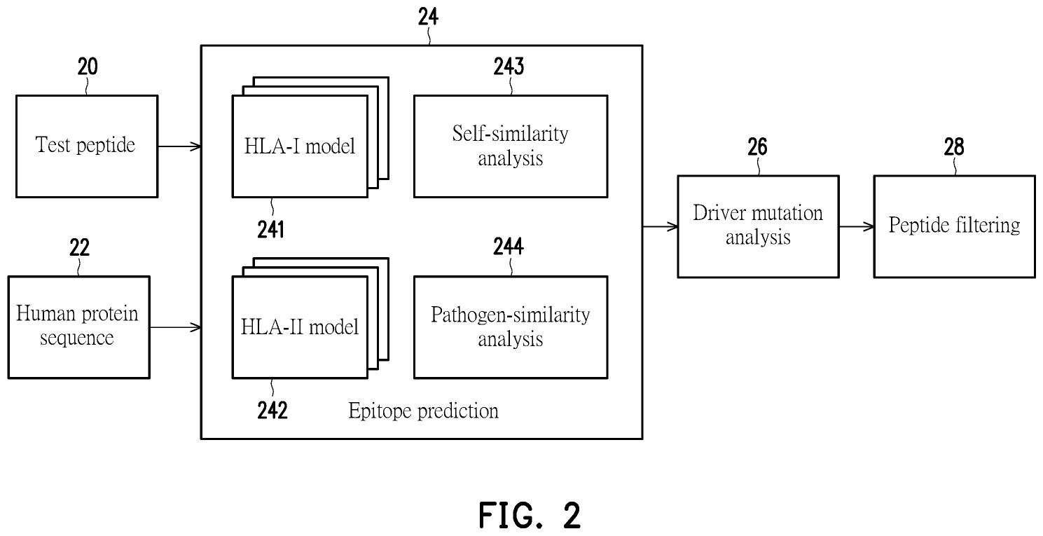

As for self-similarity, the embodiment of the disclosure conducts a search in a protein sequence database to identify wild-type sequences and variant peptides, followed by a comprehensive search across the human biological database. The step is based on the assumption that the human immune system inherently distinguishes between self and non-self, and that the proportion of peptide fragments that bind to antigens on the surface of cancerous cell, originating from self cells, can significantly impact the ability for effective immune control. The mutate peptide obtained from the protein sequence database is then utilized to search the entire human biological database to determine if the mutate peptide can bind to MHC and elicit a TCR response. FIG. 2 is a schematic diagram of tumor neoantigen prediction according to an

embodiment of the disclosure. Please refer to FIG. 2. The tumor neoantigen prediction of the embodiment is, for example, performing next-generation sequencing (NGS), that is, gene assay, on a person to be tested to extract multiple amino acid sequences in genes of the person to be tested as multiple test peptides 20 to be input to the human biological database for epitope prediction 24.

The epitope prediction 24 includes modules that provide an HLA-I model 241, an HLA-II model 242, a self-similarity analysis 243, and a pathogen-similarity analysis 244. The HLA-I model 241 is, for example, a machine learning model trained using multiple human proteins including 8-11 amino acids and multiple corresponding peptides, and is adapted to evaluate sequence of 8-11 amino acids, and the HLA-II model 242 is, for example, a machine learning model trained using multiple human proteins including 12-30 amino acids and multiple corresponding peptides, and is adapted to evaluate sequence of 12-30 amino acids, but the embodiment is not limited thereto.

After receiving the test peptides 20 input by the user, the self-similarity analysis 243 obtains, for example, multiple human protein sequences 22 from a protein sequence database such as Uniprot to be compared the human protein sequences 22 with the test peptides 20 to find multiple similar peptides that match the human protein sequences 22. The human protein sequences 22 include amino acid sequences mutated during human cell carcinogenesis. Therefore, through the comparison with the human protein sequences 22, it can be determined whether the antigen sequences of the person to be tested have any known counterparts or related sequences.

In some embodiments, the comparison is, for example, comparing the amino acid sequences as word strings, and cutting out multiple fragments containing mutated amino acids in the amino acid sequences containing the mutated amino acids using a sliding window. Through the string comparison, the amino acid sequences of the fragments are compared with the amino acid sequences of the test peptides 20 to determine whether there are matching fragments. Through comparing the amino acid sequences containing mutated amino acids, sequences related to known counterparts may be found from the test peptides 20.

Through filtering out the amino acid sequences similar to the self cells from the test peptides, the amino acid sequences that can impact effective immune control are retained for sorting and scoring. In detail, the embodiment inputs the test peptides 20 including 8-11 amino acids into multiple trained HLA-I models 241 to sort the test peptides 20 through the HLA-I models 241 and output multiple ranking results. In addition, the embodiment also inputs the test peptides 20 including 12-30 amino acids into multiple trained HLA-II models 242 to sort the test peptides 20 through the HLA-II models 242 and output multiple ranking results. For each test peptide 20, the embodiment calculates the weighted sum of rankings in the ranking results as the score of the test peptide 20.

The pathogen-similarity analysis 244 is to compare each filtered test peptide 20 with multiple known pathogen protein sequences to search for the test peptides that match the pathogen protein sequences. The pathogen-similarity analysis 244 compares the amino acid sequences of the test peptides 20 with the pathogen protein sequences, for example, through string comparison. The comparison method is the same as or similar to the method described in the previous embodiment, so no further details will be given here. In response to finding the test peptides 20 that match the pathogen protein sequences, the pathogen-similarity analysis 244 adjusts the rankings of the test peptides 20 in the ranking results (that is, the ranking results output by the HLA-I models 241 or the HLA-II models 242). In some embodiments, the pathogen-similarity analysis 244 is to move the rankings of the test peptides 20 that match the pathogen protein sequences forward or to the first position, but not limited thereto.

The ranking results and the scores of the test peptides 20 obtained via the epitope prediction 24 may be, for example, further assessed via a driver mutation analysis 26. The driver mutation analysis 26, for example, compares the filtered mutated amino acids corresponding to each test peptide 20 with multiple driver mutations of known cancers to search for the amino acids that match the driver mutations. In response to finding the amino acids that match the driver mutations, the driver mutation analysis 26 adjusts the rankings of the test peptides containing the amino acids that match the driver mutations in the ranking results. In some embodiments, the driver mutation analysis 26 moves the rankings of the test peptides 20 containing the amino acids that match the driver mutations forward, and recalculates the score of each test peptide 20 based on the changed rankings.

Finally, a peptide filtering 28 selects at least one of the test peptides as a neoantigen adapted for the person to be tested according to the score.

The tumor neoantigen prediction process may be applied to an existing immunotherapy system to predict a tumor neoantigen.

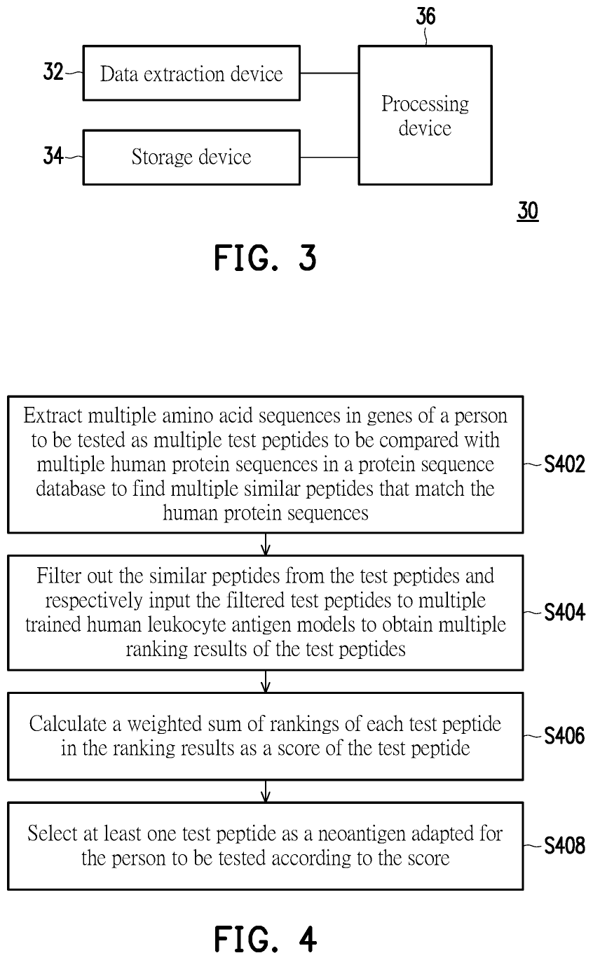

FIG. 3 is a block diagram of a tumor neoantigen prediction system according to an embodiment of the disclosure. Please refer to FIG. 3. A tumor neoantigen prediction system 30 of the embodiment of the disclosure includes a data extraction device 32, a storage device 34, and a processing device 36, and the functions thereof are described as follows.

The data extraction device 32 is, for example, a wired or wireless transmission interface such as universal serial bus (USB), RS232, Bluetooth (BT), and wireless fidelity (Wi-Fi) or a network card that supports Ethernet or wireless network standards such as 802.11g, 802.11n, and 802.11ac, and is used to extract multiple amino acid sequences in genes of a person to be tested.

The storage device 34 is, for example, any type of fixed or removable random access memory (RAM), read-only memory (ROM), flash memory, hard discs, other similar devices, integrated circuits, and a combination thereof. In some embodiments, the storage device 34 stores a protein sequence database, such as Uniprot, recorded with multiple human protein sequences.

The processing device 36 is, for example, an electronic device with computing capabilities such as a computer, a workstation, and a servers, which includes a central processing unit (CPU), a graphics processing unit (GPU), other programmable general-purpose or specific-purpose microprocessors, digital signal processors (DSPs), image signal processors (ISPs), programmable controllers, application specific integrated circuits (ASICs), programmable logic devices (PLDs), other similar devices, or a combination of the devices. In the embodiment, the processing device 36 may execute a computer program to implement the tumor neoantigen prediction method of the embodiment of the disclosure.

FIG. 4 is a schematic diagram of a tumor neoantigen prediction method according to an embodiment of the disclosure. Please refer to FIG. 3 and FIG. 4 at the same time. The method of the embodiment is applicable to the tumor neoantigen prediction system 30 of FIG. 3. The detailed process of the method of the embodiment is described below in conjunction with each device in the tumor neoantigen prediction system 30.

In Step S402, the data extraction device 32 extracts multiple amino acid sequences in genes of a person to be tested, and the processing device 36 uses the amino acid sequences extracted by the data extraction device 32 as multiple test peptides to be compared with human protein sequences in a protein sequence database stored in the storage device 34 to find multiple similar peptides that match the human protein sequences.

The data extraction device 32 is, for example, connected to a database recorded with a genetic test result of the person to be tested, thereby obtaining multiple amino acid sequences in the genes of the person to be tested. The processing device 36 compares the test peptides with the human protein sequences, for example, through string comparison or using the BLAST tool, and the embodiment does not limit the comparison manner. Through the comparison, the processing device 36 may obtain the test peptides that match the human protein sequences and the test peptides that do not match the human protein sequences, which include the sequence lengths of the test peptides, the protein identifiers of the matching human protein sequences, the alignment positions, etc.

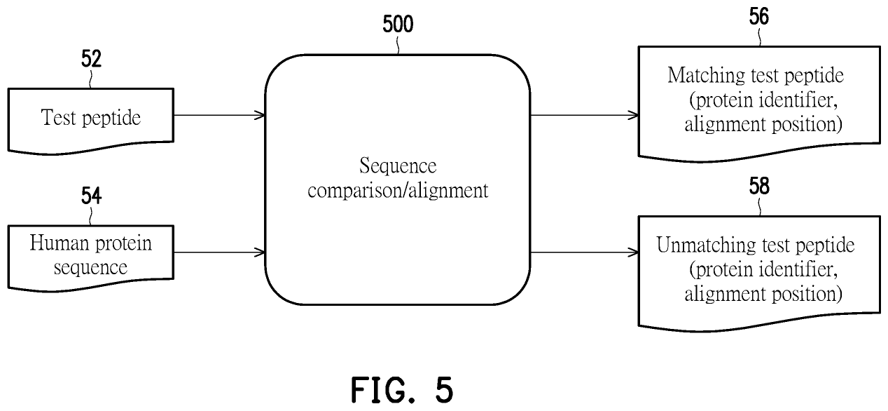

Specifically, FIG. 5 is a schematic diagram of an amino acid sequence comparison/alignment method according to an embodiment of the disclosure. Please refer to FIG. 5. In the embodiment, multiple amino acid sequences in genes of a person to be tested as test peptides 52 and multiple human protein sequences 54 provided by a protein sequence database are input to a tumor neoantigen prediction system for the procedure of a sequence comparison/alignment 500. Through string comparison or comparison using the BLAST tool, the test peptides 56 that match the human protein sequences 54 may be found from the test peptides 52, thereby outputting the matching test peptides 56, which include amino acid sequences, sequence lengths, matching protein identifiers, alignment positions, etc. On the other hand, the unmatching test peptides 56 are output, including amino acid sequences, sequence lengths, etc.

In Step S404, the processing device 36 filters out the similar peptides from the test peptides and respectively inputs the filtered test peptides to multiple trained human leukocyte antigen (HLA) models to obtain multiple ranking results of the test peptides.

In some embodiments, the processing device 36 may compare each filtered test peptide with multiple known pathogen protein sequences to search for the test peptides that match the pathogen protein sequences, and in response to finding the test peptides that match the pathogen protein sequences, the rankings of the test peptides in the ranking results are adjusted.

In some embodiments, the processing device 36 may compare the mutated amino acids corresponding to each filtered test peptide with multiple driver mutations of known cancers to search for the amino acids that match the driver mutations, and in response to finding the amino acids that match the driver mutations, and the rankings of the test peptides containing the amino acids that match the driver mutations in the ranking results are adjusted.

In Step S406, the processing device 36 calculates a weighted sum of rankings of each test peptide in the ranking results as a score of the test peptide. That is, each test peptide may have different rankings in the ranking results output by different HLA models. Through weighting the rankings and calculating the sum of the weighted rankings, the score of the test peptide may be obtained, which is used to assess whether the test peptide is suitable as a neoantigen for the person to be tested.

In Step S408, the processing device 36 selects at least one test peptide as the neoantigen adapted for the person to be tested according to the score. The selected neoantigen may be used to design a tumor neoantigen vaccine adapted to respond to gene mutations of cancerous cells of the person to be tested.

In summary, in the tumor neoantigen prediction method and the tumor neoantigen prediction system of the embodiments of the disclosure, self-similarity and pathogen-similarity analyses are performed on the amino acid sequences in the genes of the person to be tested, and in combination with the HLA models and the driver mutation analysis, the neoantigens adapted to combat the gene mutations of the cancerous cells of the person to be tested are found, which can facilitate advances in T-cell immunotherapy and personalized cancer treatment.

Although the disclosure has been disclosed in the above embodiments, the embodiments are not intended to limit the disclosure. Persons skilled in the art may make some changes and modifications without departing from the spirit and scope of the disclosure. Therefore, the protection scope of the disclosure shall be defined by the appended claims.

What is claimed is:

1. A tumor neoantigen prediction method, comprising:
extracting a plurality of amino acid sequences in genes of a person to be tested as a plurality of test peptides to be compared with a plurality of human protein sequences in a protein sequence database to find a plurality of similar peptides that match the human protein sequences;
filtering out the similar peptides from the test peptides and respectively inputting the filtered test peptides to a plurality of trained human leukocyte antigen (HLA) models to obtain a plurality of ranking results of the test peptides;
calculating a weighted sum of rankings of each of the test peptides in the ranking results as a score of the test peptide; and
selecting at least one of the test peptides as a neoantigen adapted for the person to be tested according to the score.

2. The tumor neoantigen prediction method according to claim 1, wherein after the step of obtaining the ranking results of the test peptides, the tumor neoantigen prediction method further comprises:
comparing each of the filtered test peptides with a plurality of known pathogen protein sequences to search for the test peptide that matches the pathogen protein sequences; and
adjusting the ranking of the test peptide in the ranking results in response to finding the test peptide that matches the pathogen protein sequences.

3. The tumor neoantigen prediction method according to claim 1, wherein the human protein sequences comprise amino acid sequences mutated during human cell carcinogenesis.

4. The tumor neoantigen prediction method according to claim 3, wherein after the step of obtaining the ranking results of the test peptides, the tumor neoantigen prediction method further comprises:
comparing mutated amino acids corresponding to each of the filtered test peptides with a plurality of driver mutations of known cancers to search for the amino acids that match the driver mutations; and
adjusting the ranking of the test peptide containing the amino acids that match the driver mutations in the ranking results in response to finding the amino acids that match the driver mutations.

5. The tumor neoantigen prediction method according to claim 1, wherein each of the human leukocyte antigen models comprises a machine learning model trained using a plurality of human proteins and a plurality of corresponding peptides.

6. A tumor neoantigen prediction system, comprising:
a data extraction device, extracting a plurality of amino acid sequences in genes of a person to be tested;
a storage device, storing a protein sequence database recorded with a plurality of human protein sequences; and
a processing device, coupled to the data extraction device and the storage device, and configured to:
compare the amino acid sequences extracted by the data extraction device as a plurality of test peptides with the human protein sequences in the protein sequence database to find a plurality of similar peptides that match the human protein sequences;
filter out the similar peptides from the test peptide and respectively input the filtered test peptides to a plurality of trained human leukocyte antigen models to obtain a plurality of ranking results of the test peptides;
calculate a weighted sum of rankings of each of the test peptides in the ranking results as a score of the test peptide; and
select at least one of the test peptides as a neoantigen adapted for the person to be tested according to the score.

7. The tumor neoantigen prediction system according to claim 6, wherein the processing device further compares each of the filtered test peptides with a plurality of known pathogen protein sequences to search for the test peptide that matches the pathogen protein sequences, and adjusts the ranking of the test peptide in the ranking results in response to finding the test peptide that matches the pathogen protein sequences.

8. The tumor neoantigen prediction system according to claim 6, wherein the human protein sequences comprise amino acid sequences mutated during human cell carcinogenesis.

9. The tumor neoantigen prediction system according to claim 8, wherein the processing device further compares mutated amino acids corresponding to each of the filtered test peptides with a plurality of driver mutations of known cancers to search for the amino acids that match the driver mutations, and adjusts the ranking of the test peptide containing the amino acids that match the driver mutations in the ranking results in response to finding the amino acids that match the driver mutations.

10. The tumor neoantigen prediction system according to claim 6, wherein each of the human leukocyte antigen models comprises a machine learning model trained using a plurality of human proteins and a plurality of corresponding peptides.
